(19) United States
(12) Patent Application Publication
(10) Pub. No.: US 2016/0367319 A1
(43) Pub. Date: Dec. 22, 2016

(54) STEERABLE LASER PROBE WITH PRE-CURVED STRAIGHTENING MEMBER

(71) Applicant: Medical Instrument Development Laboratories, Inc., San Leandro, CA (US)

(72) Inventors: Eduardo Villarreal, Livermore, CA (US); Erik William Peterson, Walnut Creek, CA (US); Kai Chen, Potomac, MD (US); Cristhian Octavio Reyes, San Jose, CA (US)

(21) Appl. No.: 14/743,942

(22) Filed: Jun. 18, 2015

(51) Int. Cl. A61B 18/24; A61F 9/008

(52) U.S. Cl. CPC A61B 18/24; A61F 9/00821; A61B 2017/00305

(57) ABSTRACT

A steerable laser probe including an optical fiber, a flexible tubular sleeve positioned co-axially with the flexible tubular sleeve relative to an axis, and a straightening sleeve positioned co-axially with the flexible tubular sleeve and the optical fiber relative to the axis, and positioned between the flexible tubular sleeve and the optical fiber along the axis.

### BACKGROUND

Embodiments of the invention relate to laser probes used in ophthalmologic surgeries. More particularly, embodiments of the invention relate to laser probes which are capable of bending to send light into areas typically not accessible with straight laser probes.

### SUMMARY

Some prior-art laser probes include a pre-curved, nitinol (a nickel and titanium alloy) tube and a metal (e.g., stainless steel) straightening member, which is used to straighten the nitinol tube. In such devices, the straightening member is located on the outside of the nitinol tube in a telescoping manner. Because the straightening member is positioned outside of the nitinol tube, the straightening member is larger and is made from a larger amount of material in comparison to the nitinol tube. Generally speaking, the relatively large amount of stainless steel used in the straightening member provides sufficient stiffness to the member to straighten the nitinol tube.

In other laser probes, including laser probes designed by one or more of the current Applicants, the straightening member is positioned inside a pre-curved, non-metallic, tube. In such laser probes, the pre-curved tube is made from polymeric, flexible materials, such as polyether ether ketone (PEEK). As a result of using a non-metallic material for the outer pre-curved tube, the straightening member (regardless of the amount of material from which it is made) is usually sufficiently stiff to straighten the outer pre-curved tube.

Placing a straightening member inside a pre-curved nitinol tube, however, does have drawbacks. An inner straightening member is generally smaller, and made from less material than an outer straightening member. As a result, an inner straightening member generally does not provide sufficient stiffness to straighten the pre-curved nitinol tube. Nitinol is generally stiffer than the non-metallic materials used in certain laser probes. As a consequence of using a stiffer outer tube and a less stiff inner member, full straightening of a nitinol, outer, pre-curved member with an inner straightening member is difficult, if not, impossible to achieve in practical manner.

In one embodiment, the invention provides a steerable laser probe including an optical fiber, and a first tubular sleeve. The first tubular sleeve is positioned co-axially with the optical fiber relative to an axis. The first tubular sleeve includes a first curved portion. The steerable laser probe also includes a straightening sleeve positioned co-axially with the first tubular sleeve and the optical fiber relative to the axis. The straightening sleeve includes a second curved portion. The second curved portion facilitates a more complete straightening of the first tubular sleeve in comparison to prior designs.

In another embodiment, the invention provides a steerable laser probe including an optical fiber, and a first tubular sleeve. The first tubular sleeve is positioned co-axially with the optical fiber relative to an axis. The first tubular sleeve includes a nickel alloy. The steerable laser probe also includes a straightening sleeve positioned co-axially with the first tubular sleeve and the optical fiber relative to the axis. The straightening sleeve includes a steel alloy. A second thickness of the straightening sleeve is between about 1.5 and about 3 times a first thickness of the first tubular sleeve.

Other aspects of embodiments of the invention will become apparent by consideration of the detailed description and accompanying drawings.

### BRIEF DESCRIPTION OF THE DRAWINGS

FIG. 1 is a cross-sectional view of a steerable laser probe according to one embodiment.

FIG. 2 shows a flexible tubular sleeve of the steerable laser probe.

FIG. 3 shows the steerable laser probe of FIG. 1 in a first position.

FIG. 4 shows the straightening sleeve of the steerable laser probe of FIG. 1 according to one embodiment.

FIG. 5 shows the steerable laser probe of FIG. 1 in a second position.

FIG. 6 shows the straightening sleeve of the steerable laser probe of FIG. 1 according to another embodiment.

FIG. 7 shows the steerable laser probe of FIG. 1 in a first position.

FIG. 8 shows the steerable laser probe of FIG. 1 in a second position.

FIG. 9 shows another embodiment of the steerable laser probe of FIG. 1 when the steerable laser probe is in the first position.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

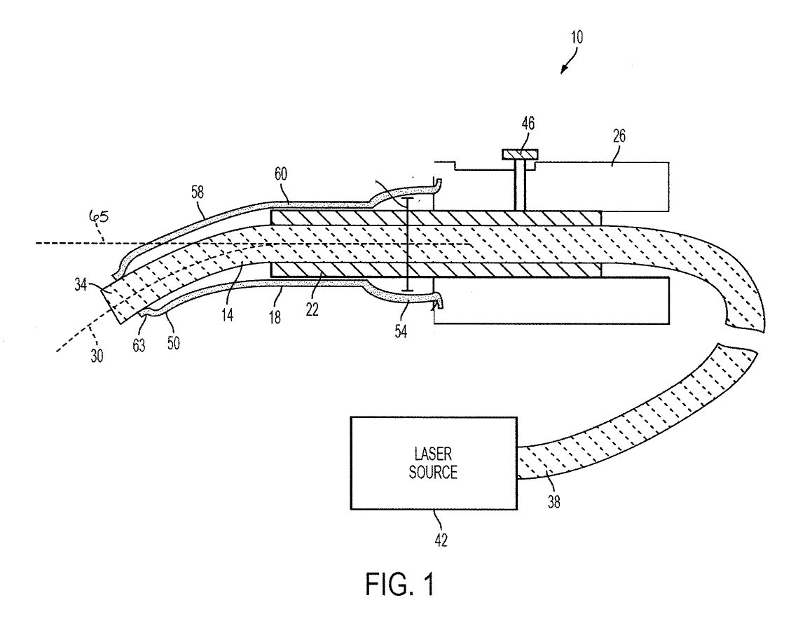

FIG. 1 illustrates one embodiment or example of a steerable laser probe 10. The steerable laser probe 10 may be used to illuminate parts of the human body that are inaccessible to external light sources. The steerable laser probe 10 may also be used, in connection with appropriate laser sources, to remove or treat tissue and organs. For example, the laser probe 10 may be used for endo-ocular photocoagulation procedures. In the embodiment shown, the steerable laser probe 10 includes an optical fiber 14, a flexible tubular sleeve 18, a straightening sleeve 22, and a handle 26. The flexible tubular sleeve 18, the optical fiber 14, and the straightening sleeve 22 are positioned co-axially with respect to one another and to an axis 30. The straightening sleeve 22 is positioned between the flexible tubular sleeve 18 and the optical fiber 14. The straightening sleeve 22 is configured to move relative to the flexible tubular sleeve 18 and optical fiber 14 to change a shape associated with the flexible tubular sleeve 18. Changing the shape of the flexible tubular sleeve 18 allows the user to direct light (e.g., laser light) or laser energy to different areas of the human body after the steerable laser probe 18 has been inserted in a cavity, organ, or tissue.

The optical fiber 14 is positioned within the flexible tubular sleeve 18 and the straightening sleeve 22. The optical fiber 14 includes a proximal end 34 and a distal end 38. The distal end 38 of the optical fiber 14 extends beyond the handle 26 and is connected to a laser source 42. The proximal end 34 of the optical fiber 14 is used to direct laser energy to a specific area. As discussed above, in some embodiments, the optical fiber 14 is used to illuminate parts of the human body, in particular parts of an eye, that are inaccessible to external light sources. The optical fiber 14 guides energy (in the form of light) from the laser source 42. Thus, the location and orientation or positioning of the optical fiber 14 determines the particular location to which laser energy from the laser source 42 is directed. Bending or curving of the optical fiber 14 changes the direction of the light and, ultimately, the location to which the light and/or laser energy is directed.

The flexible tubular sleeve 18 interacts with the optical fiber 14 to control the direction of the laser energy from the optical fiber 14. The flexible tubular sleeve 18 is secured to the handle 26 to inhibit movement of the flexible tubular sleeve 18 relative to the handle 26. The flexible tubular sleeve 18 is made from a nickel alloy material. In the illustrated embodiment, the flexible tubular sleeve is made from or composed of nitinol (e.g., a nickel and titanium alloy). The flexible tubular sleeve 18 includes a proximal end 50 and a distal end 54. The proximal end 50 of the flexible tubular sleeve 18 is approximately coterminous with the optical fiber 14. The distal end 54 of the flexible tubular sleeve 18 is fixed, at least temporarily, to the handle 26.

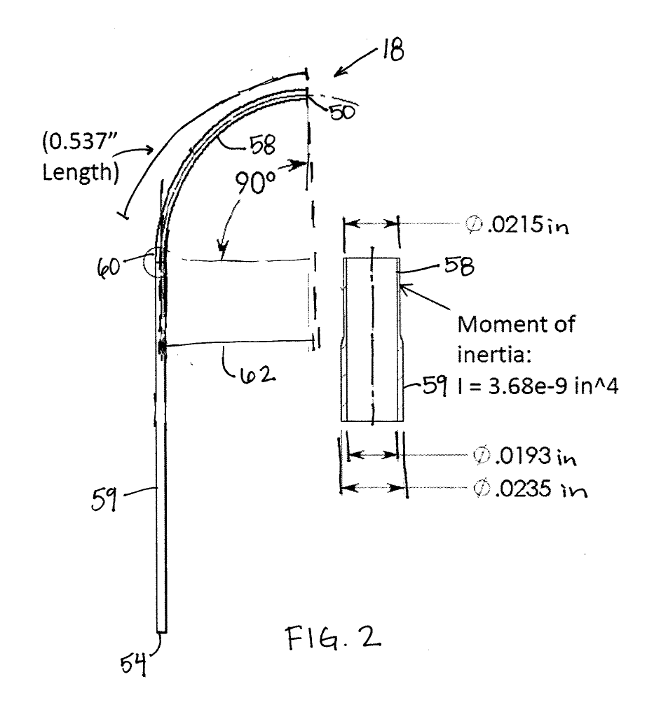

As shown in FIG. 2, the flexible tubular sleeve 18 includes a curved portion 58 and a straight portion 59. The curved portion 58 extends from a portion 60 to the proximal end 50, while the straight portion 59 extends from the portion 60 to the distal end 54. In the illustrated embodiment, the straight portion 59 has an outer diameter of 0.0235 inches and an inner diameter of 0.0193 inches, while the curved portion 58 has an outer diameter of 0.0215 inches and an inner diameter of 0.0193 inches. In other words, in the illustrated embodiment, the curved portion 58 has a smaller outer diameter than the straight portion 59 of the flexible tubular sleeve 18. Since the outer diameter decreases in the curved portion 58 of the flexible tubular sleeve 18, a thickness associated with the curved portion 58 also decreases. In the illustrated embodiment, the thickness associated with the curved portion 58 is smaller than the thickness associated with the straight portion 59 of the flexible tubular sleeve 18. In particular, the thickness associated with the curved portion 58 is approximately 0.0011 inches, and the thickness associated with the straight portion is approximately 0.0021 inches. In the illustrated embodiment, the thickness associated with the straight portion 59 is approximately two times as large as the thickness associated with the curved portion 58.

As also shown in FIG. 2, the curved portion 58 bends approximately 90°. The degree of curve or bend in the flexible tubular sleeve 18 can also be measured by determining a perpendicular distance and/or a displacement distance 62 of the curved portion 58. The displacement distance 62 is measured from an inner center of the flexible tubular sleeve 18 to a line parallel to the straight portion 49 of the flexible tubular sleeve 18 that intersects with the proximal end 50 of the flexible tubular sleeve 18. In the illustrated embodiment, the displacement distance 62 of the curved portion 58 of the flexible tubular sleeve 18 is approximately 0.37 inches. In other embodiments and depending on where the laser energy is desired, the displacement distance 62 may be different. As shown in FIG. 2, a length of the curved portion 58 is approximately 0.537 inches. The curved portion 58 of the flexible tubular sleeve 18 includes a radius of approximately ⅜ (e.g., 0.375) inches to provide the 90° bend. The radius and the length of the curved portion 58 may vary based on the displacement distance 62. It should also be understood that the flexible tubular sleeve 18 may be bent in other ways and shapes.

Referring back to FIG. 1, the curved portion 58 of the flexible tubular sleeve 18 also includes a taper 63 near the proximal end 50 of the flexible tubular sleeve 18. The taper 63 contacts and holds the optical fiber 14 so that longitudinal movement of the optical fiber 14 with respect to the flexible tubular sleeve 18 is inhibited. In addition, since both the optical fiber 14 and flexible tubular sleeve 18 are flexible, straightening and/or bending of the flexible tubular sleeve 18 causes a corresponding straightening and/or bending, respectively, of the optical fiber 14. Further, when the sleeve 18 is not straightened (or in its natural or default state), the optical fiber 14 is curved, matching the curve of the sleeve 18. When the optical fiber 14 is curved, light from the laser source may be directed at an angle from the horizontal axis 65.

The curved portion 58 of the flexible tubular sleeve 18 is anneal set at a temperature of approximately 540° C. Setting the flexible tubular sleeve 18 at such a relatively high temperature, restores the super-elastic properties to nitinol. In other words, the flexible tubular sleeve 18 is elastic such that the shape of the flexible tubular sleeve 18 may be temporarily changed. In practice, the elastic modulus for hypodermic nitinol tubing varies from 10.9 Mpsi (i.e., 10.9×10⁶ psi) to approximately 5.8 Mpsi (i.e., 5.8×10⁶ psi) based on, for example, different methods and temperatures used in the setting process.

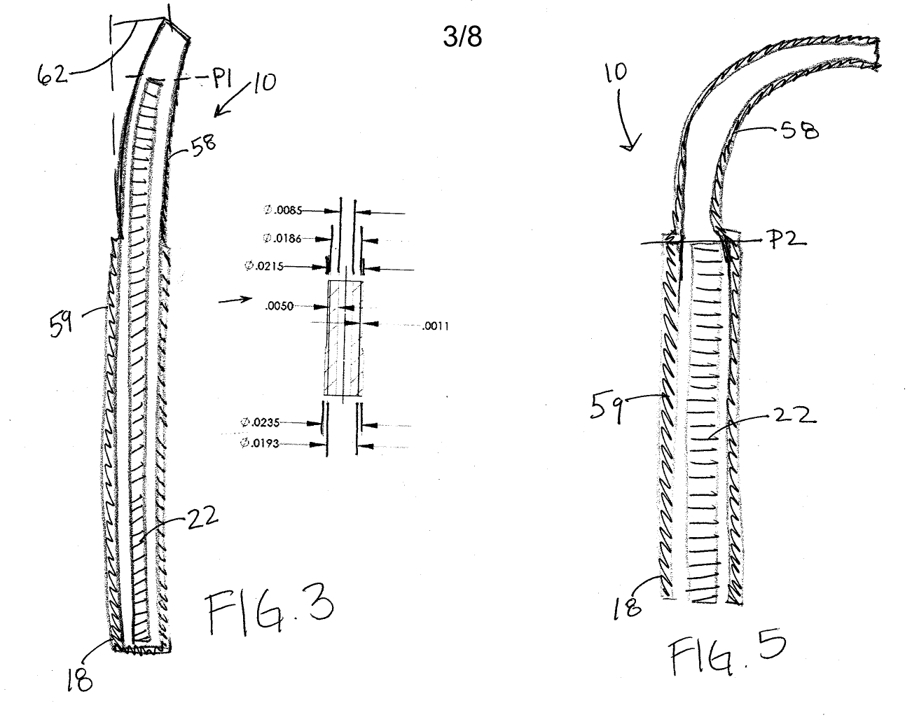

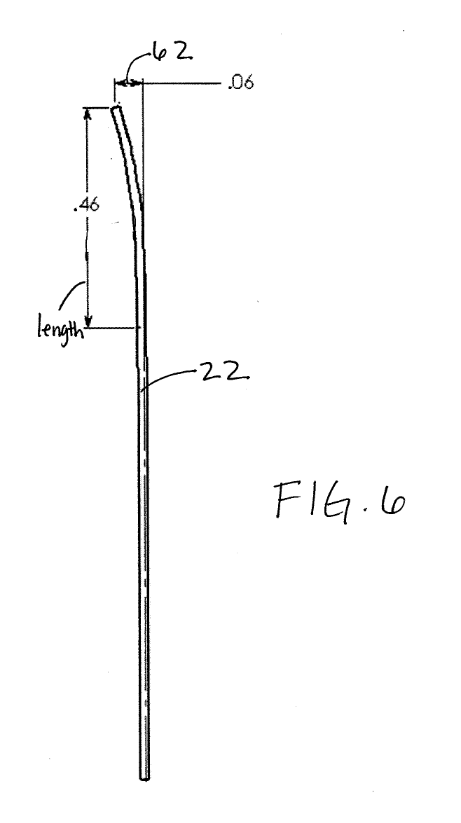

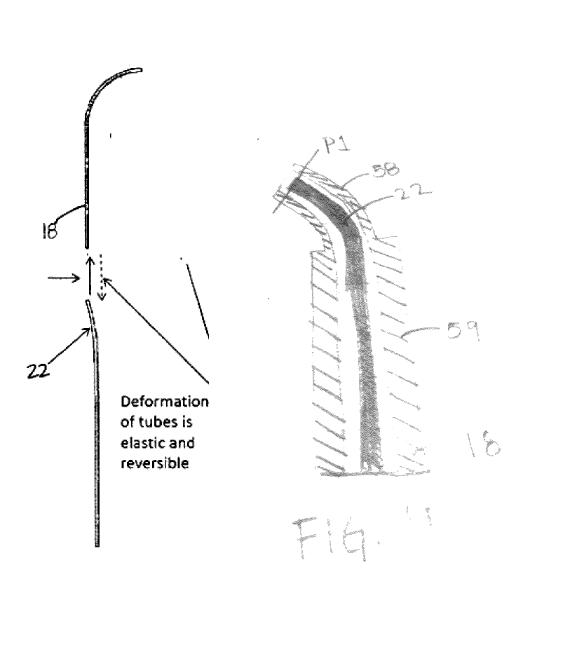

The straightening sleeve 22 is positioned between the optical fiber 14 and the flexible tubular sleeve 18. The straightening sleeve 22 is coupled to the handle 26. The handle 26 includes an actuator 64 that allows a user to control the depth of insertion of the steerable laser probe 10 and the movement of the straightening sleeve 22. In particular, the actuator 64 is connected to the straightening sleeve 22 and is configured to move the straightening sleeve 22 relative to the flexible tubular sleeve 18 and the optical fiber 14. The straightening sleeve 22 is movable, via the actuator 64, between a first portion (P1), as shown in FIGS. 3 and 7, and a second position (P2), as shown in FIGS. 5 and 8. In the first position (P1), the straightening sleeve 22 is extended toward the proximal end 50 of the flexible tubular sleeve 18. In the second position (P2), the straightening sleeve 22 retracts or moves toward the handle 26.

Preferably, the straightening sleeve 22 is made from a steel alloy. In one particular embodiment, the straightening sleeve 22 is made from stainless steel. In the illustrated embodiments, the straightening sleeve 22 is made from type 303, work-hardened stainless steel. This type of stainless steel has spring-like elastic properties and a minimum yield strength (Sy) of 140 ksi. The elastic modulus of the stainless steel used in certain embodiments is approximately 30 Mpsi (i.e., 30×10⁶ psi). In the illustrated embodiment, the straightening sleeve 22 has an inner diameter of 0.0085 inches, an outer diameter of 0.0186 inches, and an associated thickness of approximately 0.00505 inches. As shown in FIG. 3, the straightening sleeve 22 fits inside the flexible tubular sleeve 18. In the illustrated embodiment, since the flexible tubular sleeve 18 includes an inner diameter of 0.0193 inches and the straightening sleeve 22 includes an outer diameter of approximately 0.0186 inches, a space or gap of approximately 0.00035 inches separates the straightening sleeve 22 from the flexible tubular sleeve 18.

As discussed above, the straightening sleeve 22 should have sufficient stiffness to straighten the flexible tubular sleeve 18. In the illustrated embodiment, for example, a spring constant of the flexible tubular sleeve 18 can be compared to a spring constant of the straightening sleeve to analyze more quantitatively how much stiffness the straightening sleeve 22 has or provides. A spring constant of the flexible tubular sleeve 18 can be calculated by multiplying the elastic modulus and a moment of inertia associated with the flexible tubular sleeve 18. The elastic modulus associated with the flexible tubular sleeve 18, as discussed above, varies between 5.8 Mpsi and 10.9 Mpsi. The moment of inertia of the flexible tubular sleeve 18 is determined based on the inner and outer diameters of the flexible tubular sleeve 18. In particular, for the illustrated embodiment, the moment of inertia for the flexible tubular sleeve 18 is approximately 3.68×10⁻⁹ in.⁴, which yields a spring constant ranging from 0.0213 to 0.0401 lb./in. for the flexible tubular sleeve 18.

The spring constant can be analogously calculated for the straightening sleeve 22. The moment of inertia for the straightening sleeve 22 of the illustrated embodiment is approximately 5.61×10⁻⁹ in.⁴. As discussed above the elastic modulus for the straightening sleeve 22 is approximately 30 Mpsi. Accordingly, the spring constant for the straightening sleeve 22 is approximately 0.1104 lb./in. In other words, the spring constant for the straightening sleeve 22 is between 2.75 and 5.2 times the spring constant for the flexible tubular sleeve 18. Additionally, note that the elastic modulus for the straightening sleeve 22 is approximately between 275%-500% greater than the elastic modulus for the flexible tubular sleeve 18. Therefore, based on the illustrated dimensions of the straightening sleeve 22 and the flexible tubular sleeve 18, the straightening sleeve 22 can substantially straighten the flexible tubular sleeve 18 because the straightening sleeve 22 is stiffer than the flexible tubular sleeve 18.

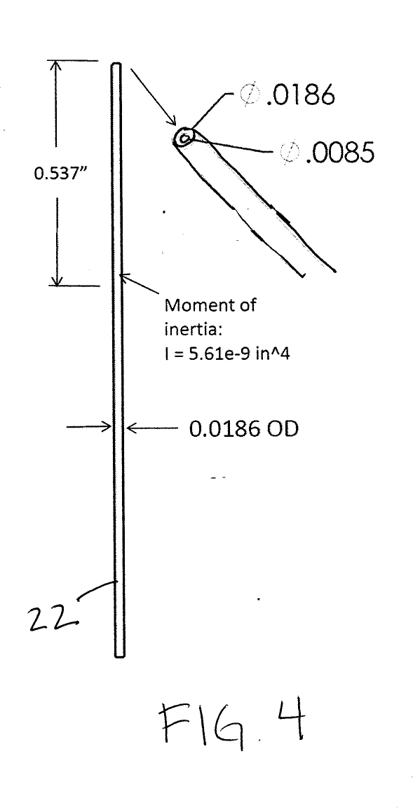

In the embodiment shown in FIGS. 3-5, the straightening sleeve 22 is substantially straight, as shown more clearly in FIG. 4. As shown in FIG. 3, in the first position P1, the straightening sleeve 22 is extended toward the proximal end 50 of the flexible tubular sleeve 18. In other words, when the straightening sleeve 22 is in the first position P1, the straightening sleeve 22 is adjacent the curved portion 58 of the flexible tubular sleeve 18. While the straightening sleeve 22 is in the first position P1, the flexible tubular sleeve 18 maintains a slight curve or bend (B). In the illustrated embodiment, the displacement distance 62 associated with the bend B of the flexible tubular sleeve 18 (when straightening sleeve 22 is in the first position P1) is approximately 0.1 inches. While the straightening sleeve 22 bends slightly, the flexible tubular sleeve 18 experiences a deflection (e.g., straightening of the curved portion 58) of approximately 0.3 inches. As shown in FIG. 5, when the straightening sleeve 22 is in the second position P2 (i.e., retracted into the handle 26), the absence of the straightening sleeve 22 allows the curved portion 58 of the flexible tubular sleeve 22 to bend or curve according to its predetermined shape as shown in FIG. 2. Said in another way, the extension of the straightening sleeve 22 to the first position P1 straightens the sleeve 18.

In some applications, the slight bend B of the flexible tubular sleeve 18 may be insignificant or otherwise have little, if any, detriment to the use of the steerable laser probe in the desired manner. In other applications, however, the slight bend B may inhibit proper use of the steerable laser probe 10. Therefore, in some embodiments, the straightening sleeve 22 incorporates a corrective feature 68. The corrective feature 68 may be created by pre-stressing the straightening sleeve 22 to create a curved portion 72. The curved portion 72 bends in a direction that is opposite the bending direction of the curved portion 58 of the flexible tubular sleeve 18. For example, while the curved portion 58 of the flexible tubular sleeve 18 bends to the right, the curved portion 72 of the straightening sleeve 22 bends to the left. Maintaining opposite bending between the flexible tubular sleeve 18 and the straightening sleeve 22 increases the spring constant of the straightening sleeve 22.

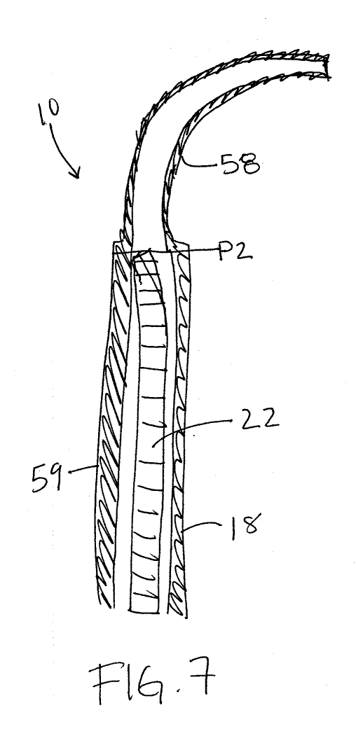

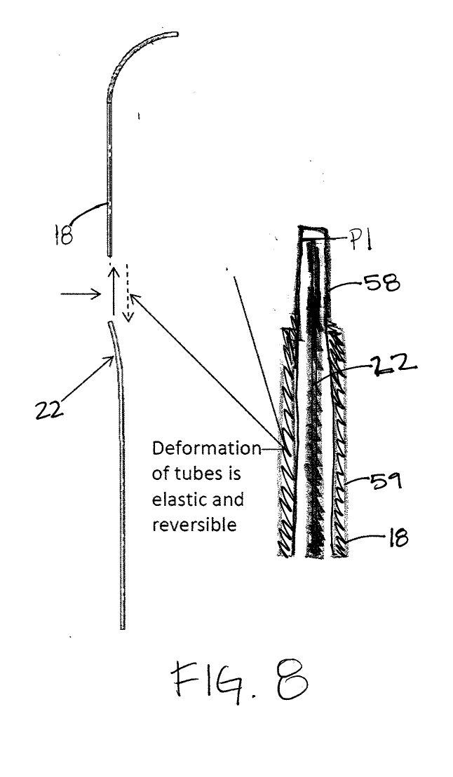

As shown in FIG. 6, the curved portion 72 has a length of approximately 0.46 inches and a displacement distance of approximately 0.06 inches. In comparison, the length of the curved portion 58 of the flexible tubular sleeve 18 is approximately 1.17 times the length of the curved portion 72 of the straightening sleeve 22. Similarly, the displacement distance of the curved portion 58 of the flexible tubular sleeve 18 is approximately 6 times larger than the displacement distance of the curved portion 72 of the straightening sleeve 22. As shown in FIG. 7, when straightening sleeve 22 (having the corrective feature 68) is in the first position, the flexible tubular sleeve 18 is straightened and contains no curve or bend because with the corrective feature 68, the spring constant of the straightening sleeve 22 increases sufficiently to withstand the bending forces of the curved portion 58 of the flexible tubular sleeve 18. As shown in FIG. 8, when the straightening sleeve 22 is in the second position, the absence of the straightening sleeve 22 (in the curved portion 58 of the flexible tubular sleeve 18) allows the flexible tubular sleeve 18 to curve according to its predetermined shape and/or natural tendencies.

The decrease in diameter of the flexible tubular sleeve 18 (from 0.0235 inches to 0.0215 inches) allows the curved portion 58 to be more flexible than the straight portion 59. The thickness of the straightening sleeve 22, on the other hand, is uniform throughout its length. The change in outer diameter of the flexible tubular sleeve 18 allows the straightening sleeve 22 to straighten the curved portion 58 of the flexible tubular sleeve 18, but inhibits the curved portion 72 of the straightening sleeve 22 from bending the straight portion 59 of the flexible tubular sleeve 18. In other words, while the thickness of the straightening sleeve 22 is approximately 2.4 times larger than the thickness of the flexible tubular sleeve 18 at the straight potion 59, the thickness of the straightening sleeve 22 is approximately 4.59 times larger than the thickness of the flexible tubular sleeve 18 at the curved portion 58. The increase in thickness ratio (straightening sleeve 22 to flexible tubular sleeve 18) is related to the stiffness and straightening force provided by the straightening sleeve 22 to the flexible tubular sleeve 18, such that the straightening sleeve 22 can change the shape (e.g., straighten) the curved portion 58 of the flexible tubular sleeve 18, but not the straight portion 59 of the flexible tubular sleeve 18.

In some embodiments, the slight bend B of the flexible tubular sleeve 18 may be desired by a user. In such instances, the curved portion 72 of the straightening sleeve 22 includes a bend that is more heavily accentuated such that when the straightening sleeve 22 is in the first position, the flexible tubular sleeve 18 is bent in the direction of the bend B, not in the direction of the curved portion 58, as shown in FIG. 9. In other words, the curved portion 72 of the straightening sleeve 22 may serve not only to straighten the flexible tubular sleeve 18, but may create a bend in the opposite direction of the curved portion 58 of the flexible tubular sleeve 18.

In other embodiments, the flexible tubular sleeve 18 is weakened by laser cutting lines or ridges along the length of the curved portion 58 of the flexible tubular sleeve 18. The curved portion 58 may be weakened in addition to or instead of decreasing the outer diameter of the curved portion 58 of the flexible tubular sleeve 18. When the curved portion 58 is weakened by creating ridges (e.g., partially cutting longitudinal lines along the curved portion 58), the straightening sleeve 22 can provide sufficient stiffness to straighten the curved portion 58 of the flexible tubular sleeve when the straightening sleeve 22 is in the first position.

A user inserts the steerable laser probe 10 into an area, such as a cavity in the eye, while the straightening sleeve 22 is in the first position. The user changes the angle of the projected laser energy from the optical fiber 14 by retracting the straightening sleeve 22 to the second position. When the steerable laser probe 10 needs to be removed from the area, the straightening sleeve 22 is moved back to the first position to inhibit the optical fiber 14 from curving. The steerable laser probe 10 is more easily removed from the area when in a straightened state.

In prior-art devices, steerable laser probes often position a flexible tube inside a rigid tube. The flexible tube moves from a retracted position to an extended position. In the retracted position, the flexible tube is co-axially positioned inside the rigid tube and inhibited from curving. However, in the extended position, the flexible tube moves past the rigid tube and is able to bend. However, as the flexible tube bends or curves, the flexible tube also experiences longitudinal displacement. A user has to account for the longitudinal displacement to direct the light from an optical fiber in a desired direction and such steerable probes require the user to adjust the depth of insertion of the probe.

Since the steerable laser probe 10 includes the straightening sleeve 22 in between the optical fiber 14 and the flexible tubular sleeve 18, and the straightening sleeve 22 retracts relative to the optical fiber 14 and the flexible tubular sleeve 18, the flexible tubular sleeve 18 bends or curves without experiencing longitudinal displacement. As a consequence, the steerable laser probe 10 provides a user with an easy way to direct laser energy from the optical fiber 14 to a desired location without requiring adjustment of the depth of insertion of the steerable laser probe 10 due to longitudinal displacement.

Thus, embodiments provide, among other things, a steerable laser probe that inhibits longitudinal displacement of the optical fiber while changing the angle at which light is directed. Various features and advantages of the invention are set forth in the following claims.

What is claimed is:

1. A steerable laser probe comprising:
an optical fiber;
a first tubular sleeve positioned co-axially with the optical fiber relative to an axis, the first tubular sleeve including a straight portion having a first thickness and a first curved portion having a second thickness, the second thickness being less than the first thickness; and
a straightening sleeve positioned co-axially with the first tubular sleeve and the optical fiber relative to the axis, the straightening sleeve including a second curved portion.

2. The steerable laser probe of claim 1, wherein the straightening sleeve is positioned between the first tubular sleeve and the optical fiber along the axis.

3. The steerable laser probe of claim 1, wherein the first tubular sleeve and the straightening sleeve are elastic.

4. The steerable laser probe of claim 1, wherein the straightening sleeve is movable along the axis relative to the first tubular sleeve and the optical fiber, wherein the straightening sleeve is movable between a first position and a second position, and wherein, when the straightening sleeve is in the first position, the straightening sleeve substantially straightens a portion of the first tubular sleeve and, when the straightening sleeve is in the second position, the absence of the straightening sleeve allows the portion of the first tubular sleeve to curve.

5. The steerable laser probe of claim 4, further comprising a handle having a mechanism that activates movement of the straightening sleeve between the first position and the second position.

6. The steerable laser probe of claim 4, wherein the optical fiber curves according to the first curved portion of the first tubular sleeve when the straightening sleeve is in the second position, and wherein the first curved portion is bent in a first direction and the second curved portion is bent in a second direction, and wherein the first direction is opposite the second direction.

7. The steerable laser probe of claim 6, wherein when the straightening sleeve is in the first position, part of the first curved portion of the first tubular sleeve is curved with respect to the straight portion of the first tubular sleeve.

8. The steerable laser probe of claim 7, wherein the part of the first curved portion is curved in the first direction when the straightening sleeve is in the first position.

9. The steerable laser probe of claim 7, wherein the part of the first curved portion is curved in the second direction when the straightening sleeve is in the first position.

10. The steerable laser probe of claim 1, wherein the first tubular sleeve is composed of nitinol and the straightening sleeve is composed of stainless steel.

11. The steerable laser probe of claim 1, wherein a length of the first curved portion of the first tubular sleeve is between about 1 and about 2 times a length of the second curved of the straightening sleeve.

12. The steerable laser probe of claim 11, wherein the length of the first curved portion of the first tubular sleeve is about 1.2 times longer than the length of the second curved portion of the straightening sleeve.

13. The steerable laser probe of claim 1, wherein the first curved portion of the first tubular sleeve has a displacement distance that is between two and ten times longer than a displacement distance for the second curved portion of the straightening sleeve.

14. A steerable laser probe comprising:
an optical fiber;
a first tubular sleeve positioned co-axially with the optical fiber relative to an axis, the first tubular sleeve composed of a nickel alloy, the first tubular sleeve having a first portion of a first thickness and a second portion of a second thickness; and
a straightening sleeve positioned co-axially with the first tubular sleeve and the optical fiber relative to the axis, the straightening sleeve composed of steel alloy,
wherein a third thickness of the straightening sleeve is between about 1.5 and about 3 times a first thickness of the first tubular sleeve.

15. The steerable laser probe of claim 14, wherein the nickel alloy includes nitinol.

16. The steerable laser probe of claim 14, wherein the steel alloy includes stainless steel.

17. The steerable laser probe of claim 14, wherein the straightening sleeve is positioned between the first tubular sleeve and the optical fiber.

18. The steerable laser probe of claim 14, wherein the straightening sleeve is movable along the axis relative to the first tubular sleeve and the optical fiber, wherein the straightening sleeve is movable between a first position and a second position, and wherein, when the straightening sleeve is in the first position, the straightening sleeve substantially straightens a portion of the first tubular sleeve and, when the straightening sleeve is in the second position, the absence of the straightening sleeve allows the portion of the first tubular sleeve to curve.

19. The steerable laser probe of claim 14, wherein an outer diameter of the first tubular sleeve is approximately 0.0215 inches.

20. The steerable laser probe of claim 14, wherein a first thickness of the first tubular sleeve is approximately between 0.001 and 0.004 inches, and wherein a second thickness of the straightening sleeve is between about 0.0030 and about 0.006 inches.

21. The steerable laser probe of claim 14, wherein the straightening sleeve is approximately 2.5 times thicker than the first tubular sleeve.

22. The steerable laser probe of claim 14, wherein the first tubular sleeve includes a distal end and a proximal end, the proximal end of the first tubular sleeve being coupled to a handle, and wherein a thickness of the first tubular sleeve decreases near the distal end of the first tubular sleeve.

23. The steerable laser probe of claim 14, wherein a spring constant associated with the straightening sleeve is approximately 3 times larger than the spring constant associated with the first tubular sleeve.

24. The steerable laser probe of claim 18, wherein, when the straightening sleeve is in the first position, the straightening sleeve bends a portion of the first sleeve toward a first direction, and when the straightening sleeve is in the second position, the absence of the straightening sleeve allows the portion of the first sleeve to bend toward a second direction, and wherein the first direction is opposite the second direction.
